(19) United States
(12) Patent Application Publication
(10) Pub. No.: US 2009/0099453 A1
(43) Pub. Date: Apr. 16, 2009

(54) STAND-ALONE CW DOPPLER PROBE INTERFACE FOR PHASED ARRAY ULTRASOUND SYSTEM

(76) Inventor: Kjell Kristoffersen, Oslo (NO)

Correspondence Address: DEAN D. SMALL;THE SMALL PATENT LAW GROUP LLP, 225 S. MERAMEC, STE. 725T, ST. LOUIS, MO, 63105, US

(21) Appl. No.: 11/873,193

(22) Filed: Oct. 16, 2007

(51) Int. Cl. A61B 8/00

(52) U.S. Cl. 600/459

(57) ABSTRACT

A stand-alone continuous wave (CW) Doppler probe (CW probe) comprises a scanning end that has a single transmit element and a single receive element. A connector has probe connecting elements configured to interface with port connecting elements of a probe port that is provided on an ultrasound system. The port connecting elements are configured to connect with system channels of the ultrasound system. Transmit circuitry connects a first set of the probe connecting elements in parallel, and the transmit circuitry is interconnected with the transmit element. Receive circuitry connects a second set of the probe connecting elements in parallel, and the receive circuitry is interconnected with the receive element.

### BACKGROUND OF THE INVENTION

This invention relates generally to ultrasonic imaging, and more particularly to continuous wave (CW) Doppler ultrasound probes.

A CW Doppler probe may be used for evaluation of blood flow, for example in the human heart. CW Doppler probes have two transducer elements that are typically arranged as a split disc with one element used as an ultrasonic wave transmitter and the other element functioning as a reflected wave receiver. Blood flow information is obtained without obtaining imaging data. The CW Doppler probe has increased sensitivity relative to a phased array probe used for imaging, and it may be desirable to use the CW Doppler probe in anatomical positions when it is difficult to obtain satisfactory images with a phased array probe.

The CW Doppler probe has some operational requirements that are different than phased array probes. The CW Doppler probe requires a larger transmit current and a lower receiver noise floor than typically available from a single ultrasound system channel as provided through the port for the phased array probes. Therefore, the system provides a separate port for the CW Doppler probe and the system houses the components that are needed to operate the CW Doppler probe. Therefore, the CW Doppler probe can only be used with systems having the optional hardware and probe port installed. In addition, as systems become smaller, less room is available for a dedicated CW Doppler probe port, as well as the required additional circuitry. For example, with very small handheld and laptop sized systems, a single phased array probe port may be provided.

Therefore, a need exists for an improved interface between the CW Doppler probe and the ultrasound system.

### BRIEF DESCRIPTION OF THE INVENTION

In one embodiment, a stand-alone continuous wave (CW) Doppler probe (CW probe) comprises a scanning end that has a single transmit element and a single receive element. A connector has probe connecting elements configured to interface with port connecting elements of a probe port that is provided on an ultrasound system. The port connecting elements are configured to connect with system channels of the ultrasound system. Transmit circuitry connects a first set of the probe connecting elements in parallel, and the transmit circuitry is interconnected with the transmit element. Receive circuitry connects a second set of the probe connecting elements in parallel, and the receive circuitry is interconnected with the receive element.

In another embodiment, a stand-alone CW probe comprises a scanning end that has a single transmit element and a single receive element. The CW probe also has a connector that has a plurality of probe connecting elements that are configured to interface with a plurality of port connecting elements of a probe port provided on an ultrasound system. The probe port is configured to accept a connector of a phased array probe. The plurality of port connecting elements is configured to connect with system channels of the ultrasound system.

In yet another embodiment, a method for powering a stand-alone CW probe using a phased array connector comprises determining respective areas of a single transmit element and a single receive element within a CW probe. N transmit channels are connected in parallel within the CW probe to form a transmit line that is interconnected with the transmit element. N is based at least on the area of the transmit element. M receive channels are connected in parallel within the CW probe to form a receive line that is interconnected with the receive element. M is based at least on a dynamic range associated with processing receive signals from the receive element, and the transmit and receive channels are connected through a probe port on an ultrasound system that is configured to interface with phased array probes.

### BRIEF DESCRIPTION OF THE DRAWINGS

FIG. 1 is a schematic block diagram of an ultrasound system and a stand-alone continuous wave (CW) Doppler probe (CW probe) formed in accordance with an embodiment of the present invention.

FIG. 2 illustrates a stand-alone CW probe formed in accordance with an embodiment of the present invention.

FIG. 3 illustrates a schematic diagram of transmit/receive circuitry used to operate the CW probe in accordance with an embodiment of the present invention.

FIG. 4 illustrates an exemplary miniaturized ultrasound system that may be connected to the CW probe through a phased array probe port in accordance with an embodiment of the present invention.

FIG. 5 illustrates a hand carried or pocket-sized ultrasound imaging system that may be connected to the CW probe through a phased array probe port in accordance with an embodiment of the present invention.

FIG. 6 illustrates a console ultrasound imaging system provided on a movable base that may be connected to the CW probe through a phased array probe port in accordance with an embodiment of the present invention.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like). Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

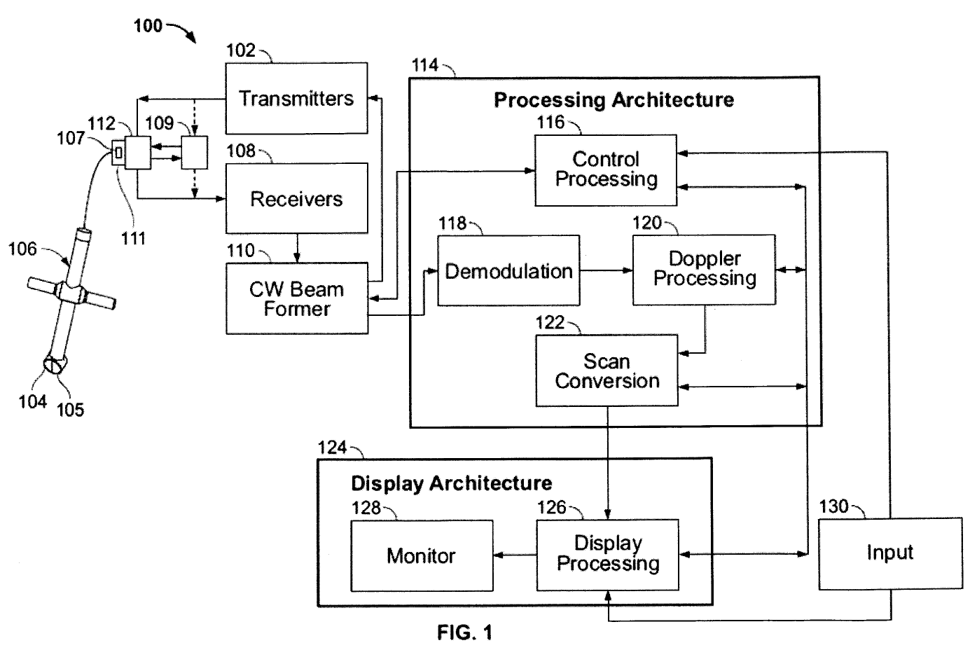

FIG. 1 is a schematic block diagram of an ultrasound system 100 and a stand-alone continuous wave (CW) Doppler probe (CW probe) 106. The CW probe 106 interfaces with the system 100 through a probe port 112 that also interfaces with phased array probes. The CW probe 106 has a transmit element 104 and receive element 105 (e.g. piezoelectric elements), as well as integral transmit/receive circuitry 107 within a connector shell 111. Previously, the transmit/receive circuitry 107 was installed and provided within the system 100 rather than within the CW probe connector or connector shell 111, and a dedicated CW probe port (not shown) was required to connect the CW probe 106 to the system 100. In an alternative embodiment, transmit/receive circuitry 109 may be provided within the system 100 or may be removably connected (e.g. removable adapter) while the CW probe 106 is connected to the phased array probe port 112.

A CW beamformer 110 within the system 100 controls transmitters 102 to drive the transmit element 104 to emit pulsed ultrasonic signals into a body. The ultrasonic signals are back-scattered from structures in the body, like blood cells or muscular tissue, to produce echoes that return to receive element 105. The echoes are received by receivers 108 in the system 100. Processing architecture 114 comprises a control processing module 116, a demodulation module 118, a Doppler processing module 120, and a scan conversion module 122. Display architecture 124 comprises a display processing module 126 and a monitor 128. Input 130 may comprise a keyboard, trackball, microphone, switches, knobs, control keys, and the like.

The port 112 is connected to the output of the transmitters 102 and the inputs of the receivers 108. The outputs of the receivers 108 are input to the CW beamformer 110. The CW beamformer 110 is further connected to the input of transmitters 102, to the control processing module 116 and the input of the demodulation module 118 in processing architecture 114. The CW beamformer 110 outputs a sum of individually delayed versions of the input signals to the demodulation module 118.

In the processing architecture 114, the output of the demodulation module 118 is connected to an input of the Doppler processing module 120. Control processing module 116 interfaces to the Doppler processing module 120 and scan conversion module 122, to the display processing module 126 in the display architecture 124, and to the input 130. An output of the Doppler processing module 120 is connected to an input of scan conversion module 122. An output of scan conversion module 122 is connected to an input of the display processing module 126 in display architecture 124, and the output of the display processing module 126 is connected to the input of the monitor 128. An output of the input 130 is connected to the display processing module 126.

To generate a transmitted ultrasound beam, the control processing module 116 sends command data to the CW beamformer 110 that tells the CW beamformer 110 to generate transmit parameters to create a transmit beam. The transmit parameters are sent from the CW beamformer 110 to the transmitters 102. The transmitters 102 use the transmit parameters to properly encode transmit signals to be sent to the CW probe 106 through the port 112. The transmit signals are identical with respect to each other. The transmit signals excite the transmit element 104 to emit ultrasound waves. As a result, a transmitted beam of ultrasound energy is formed in a subject when the transmit element 104 is acoustically coupled to the subject by using, for example, ultrasound gel.

The ultrasound waves are backscattered off of tissue and blood samples within the structure. The backscattered wave arrives at the receive element 105. The receive element 105 is responsive to the backscattered wave and converts the ultrasound energy from the backscattered wave into a received electrical signal that is routed through the port 112, which distributes the electrical signal to the different receivers 108. The receivers 108 may amplify and possibly also digitize the received signals and may provide other functions.

The received signals are sent to the CW beamformer 110. The CW beamformer 110 applies the same delay to each of the received signals and sums the delayed received signals, which are then passed to the processing architecture 114. Demodulation module 118 performs demodulation on the received beam signals to create pairs of I and Q demodulated data values. Demodulation is accomplished by comparing the phase and amplitude of the received beam signals to a reference frequency. The I and Q demodulated data values preserve the phase and amplitude information induced by Doppler shifts in the received signals.

The demodulated data is transferred to Doppler processing module 120. The Doppler processing module 120 uses standard techniques such as discrete Fourier transform (DFT) processing to generate a set of spectral Doppler data that may be displayed on the monitor 128. It should be understood that the processing architecture 114 is configured to process ultrasound data according to a plurality of selectable ultrasound modalities and types of ultrasound probes, such as phased array probes, which may also be connected to the system 100 through the port 112.

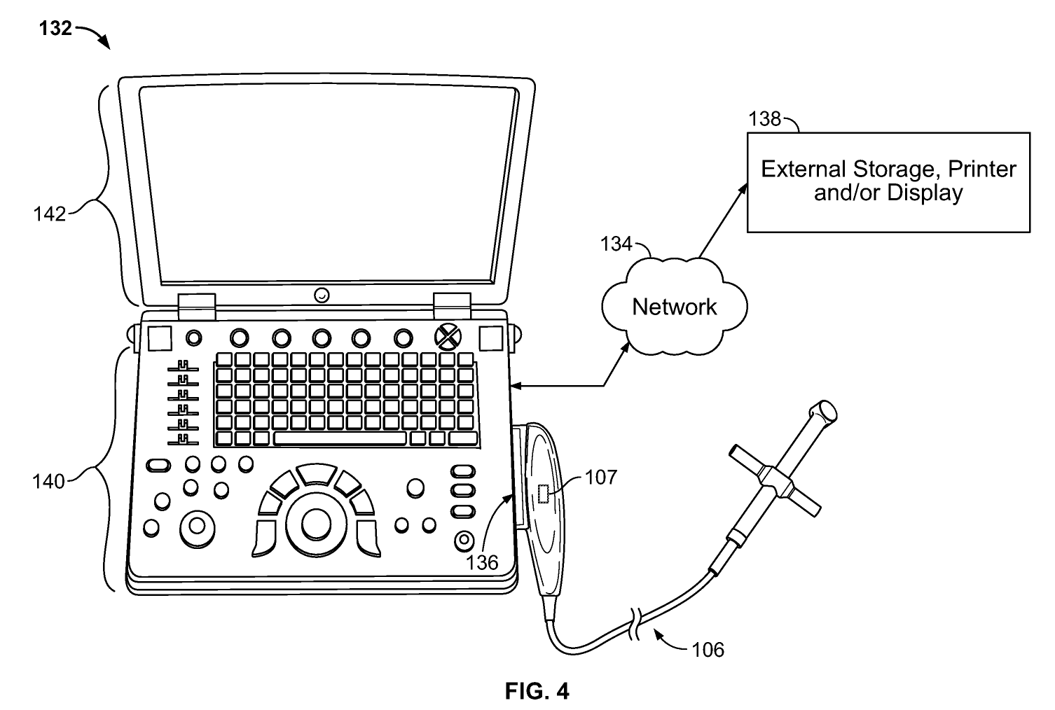

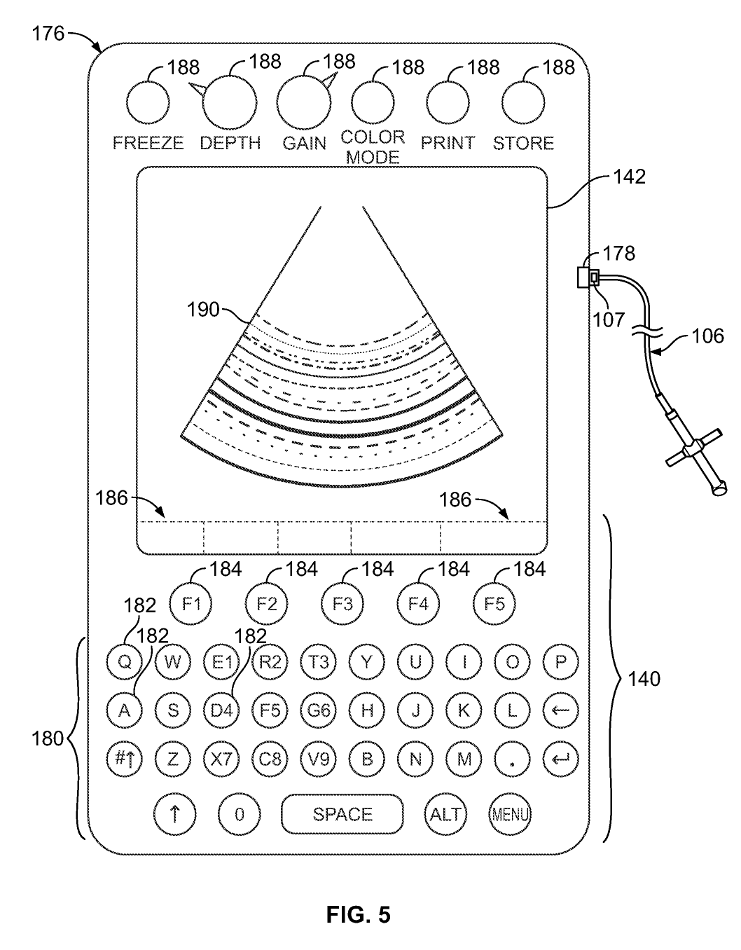

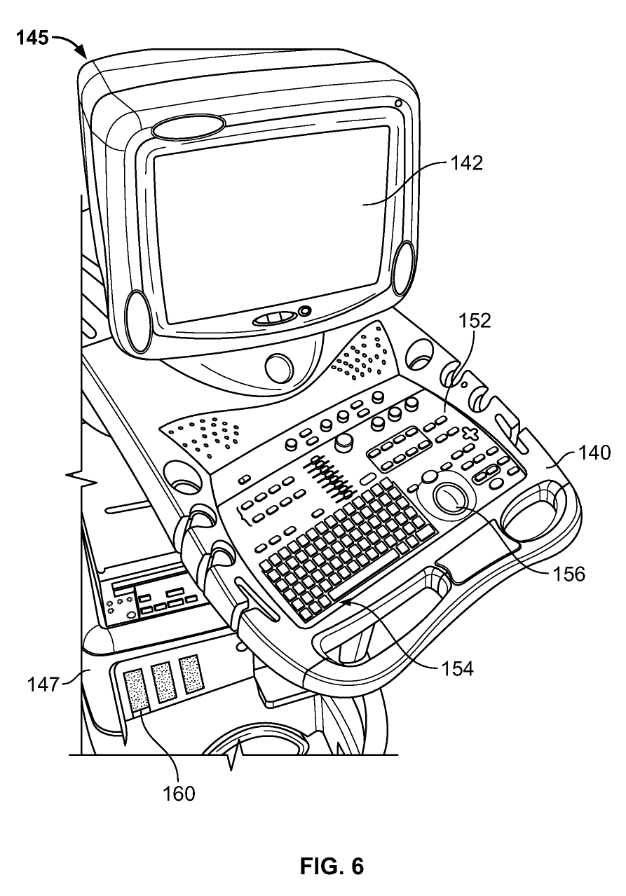

The generalized ultrasound system 100 of FIG. 1 may be embodied in a small-sized system, such as laptop computer or pocket sized system as well as in a larger cart-based system. FIGS. 4 and 5 are directed towards smaller-sized systems, while FIG. 6 is directed towards relatively larger systems. FIGS. 4-6 are discussed further below.

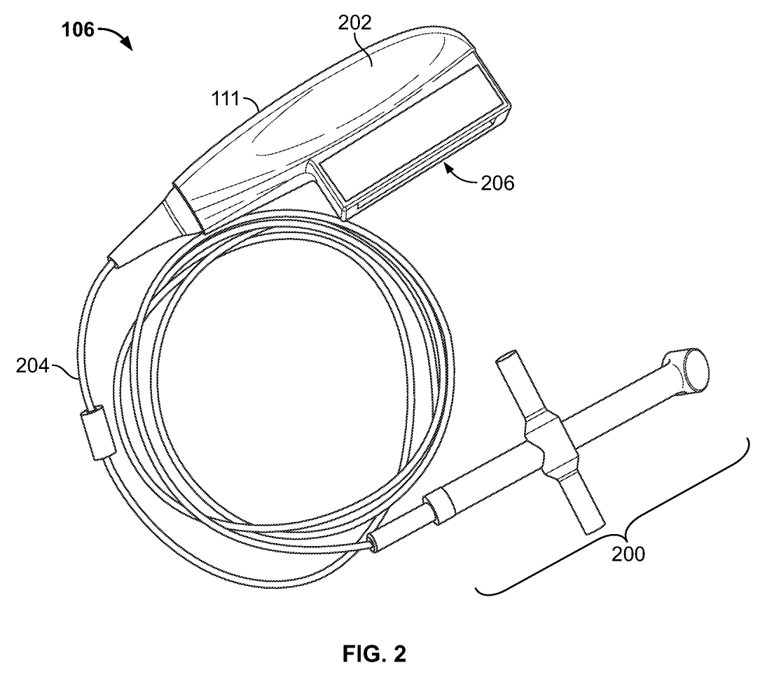

FIG. 2 illustrates a stand-alone CW probe 106. The CW probe 106 has a scanning end 200 that houses the transmitter and receiver elements 104 and 105 as shown in FIG. 1. The scanning end 200 is connected to probe connector 202 with a cable 204. Wires within the cable 204 convey signals between the scanning end 200 and the connector 202. An outer housing or the probe connector shell 111, which typically comprises one or more pieces, encloses the connector 202. In one embodiment, the transmit/receive circuitry 107 of FIG. 1 is housed within the connector 202. In another embodiment, the transmit/receive circuitry 107 may be housed within the scanning end 200 and/or within the cable 204, or may be separated and located at different areas within the connector shell 111 of the CW probe 106. For example, transmit portions of the transmit/receive circuitry 107 may be housed within the scanning end 200 while receiver portions of the transmit/receive circuitry 107 are housed within the connector 202.

The connector 202 is configured to be attached to the port 112 on the system 100 that also accepts phased array probes. Therefore, the connector 202 has a plurality of probe connecting elements 206, such as pins, that interconnect with corresponding port connecting elements (not shown), such as holes, within the port 112. In another embodiment, the probe connecting elements 206 may be holes for receiving pins (the corresponding port connecting elements) of the port 112.

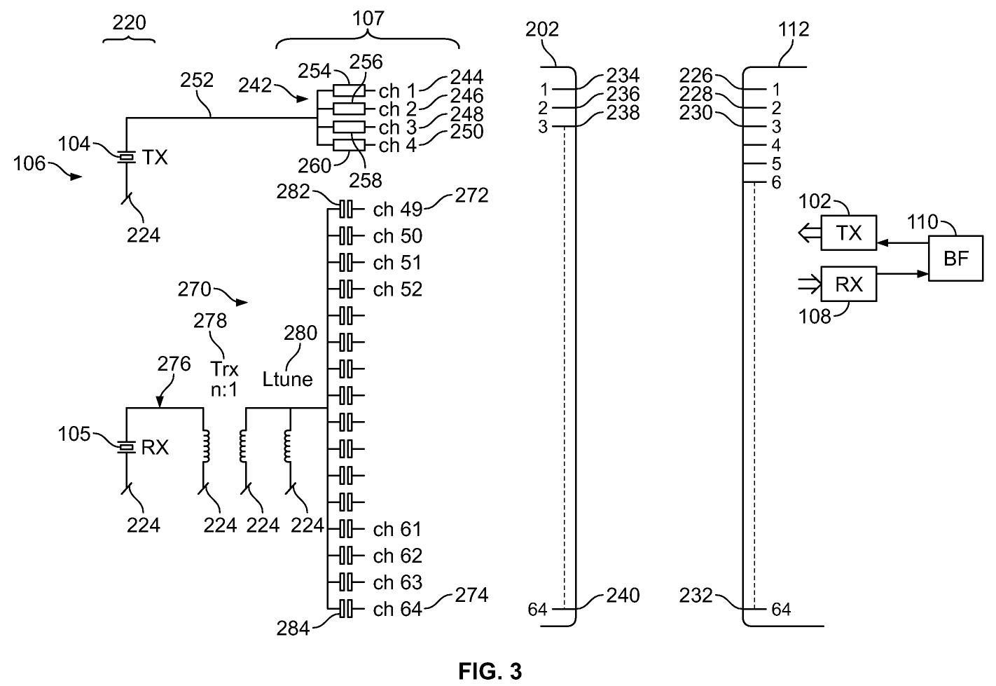

FIG. 3 illustrates a schematic diagram of the transmit/receive circuitry 107. Interconnection within the stand-alone CW probe 106 and the relationship to the port 112 within the system 100 of FIG. 1 is also shown. Portion 220 of the CW probe 106 indicates the transmit element 104 and the receive element 105. The transmit and receive elements 104 and 105, as well as other circuitry discussed below, may be connected to a common ground 224.

In this example, the system 100 has 64 system channels, each of which corresponds to one of 64 port connecting elements within the port 112. As used herein, the term system channel refers to the circuitry as well as the electrical signals associated with the system channel. First, second, third through 64^{th} port connecting elements 226, 228, 230 through 232 are indicated and correspond to first, second, third through 64^{th} probe connecting elements 234, 236, 238 through 240 in the connector 202 of the CW probe 106. It should be understood that the system 100 may have more or less channels, such as 32 channels, and that the number of port and probe connecting elements may be different based on different system platforms. For example, a system 100 may use a multiplexer (not shown) to increase the number of system channels used for both transmit and receive operations without increasing the number of connecting elements. In one embodiment, the connector 202 may have a smaller number of probe connecting elements 234-240, corresponding to the number of system channels used by the transmit/receive circuitry 107.

As discussed previously, the CW probe 106 has two elements, the transmit and receive elements 104 and 105, while an array probe has an array of many elements that may be used for transmit, receive, or both. The areas (or sizes) of transmit and receive elements 104 and 105 are much larger than areas of individual elements of the array probe. The larger area or size of the elements changes such properties as electrical impedance, voltage noise level requirements and electrical driving current requirements, and thus the transmit and receive elements 104 and 105 may not be directly connected to single channels of the system 100, as is done with array probes. For example, if the transmit and receive elements 104 and 105 are 20 times larger than a typical element used in an array probe with the system 100, the receive side of the CW probe 106 has an electrical impedance that is 20 times lower, resulting in a requirement for the receivers 108 of the system 100 to have a lower voltage noise level of approximately square root of 20 (i.e., sqrt(20)) times the voltage noise level needed for the array probe. The transmit side of the CW probe 106 requires approximately 20 times the current capability of a single system channel of the system 100.

In general, a first plurality of system channels are operated as transmit channels and are connected in parallel within a transmit portion of the transmit/receive circuitry 107. During transmit, the channels within the first plurality of system channels are programmed to provide identical outputs. A second plurality of system channels are operated as receive channels and are connected in parallel within a receive portion of the transmit/receive circuitry 107. During receive, the CW beamformer 110 applies identical delays to the received signals before summing and outputting the signals.

Transmit circuitry 242 will be discussed first. In this example, first, second, third and fourth system channels 244, 246, 248 and 250 are connected together in parallel. The first through fourth system channels 244-250 are conveyed from the transmitters 102 of the system 100 through separate probe connector elements 234-240. This configuration increases the current to drive the relatively larger transmit element 104. Transmit line 252 conveys the signal from the parallel system channels 244-250 of the transmit circuitry 242 to the transmitter element 104. Optionally, first, second, third and fourth resistors 254, 256, 258 and 260 are connected in series with the first through fourth system channels 244-250, respectively, to provide short circuit protection for the transmitters 102. The first through fourth resistors 254-260 are positioned upstream of the transmit line 252 and the transmit element 104. Although the first through fourth resistors 254-260 are shown, other circuit elements may be used to provide short circuit protection. During operation, all of the transmit channels (e.g. first through fourth system channels 244-250) are programmed to have the same delay. In other words, the transmitters 102 output the same transmit signal on all of the system channels 244-250.

By placing the transmit channels in parallel, a higher current capability is obtained and a cleaner transmit waveform having lower noise is achieved because the output voltage (conveyed on transmit line 252) is the average of four contributions. Therefore, the noise reduces by a factor of sqrt(4), assuming uncorrelated noise between the transmit channels. More or less than four transmit channels may be used, depending upon the current capability and a predetermined voltage noise level (e.g. average voltage noise level) associated with the transmit signal of one transmit channel.

Receive circuitry 270 will now be discussed. Forty-ninth through sixty-fourth system channels 272 through 274 are connected together in parallel. Receive line 276 conveys the receive signal from the receive element 105 to the 16 parallel system channels 272-274. Again, during operation, all of the receive channels are programmed to have the same delay (such as in the CW beamformer 110).

By placing the receive channels in parallel, the effective voltage noise level is reduced by a factor of the square root of the number of parallel channels, in this example, sqrt(16). Therefore, if the voltage noise level of one receive channel is 1.3 nanovolt/root Hertz (nV/rtHz), the voltage noise level after paralleling is reduced to 1.3/sqrt(16)=0.325 nV/rtHz. By paralleling the receive channels, the combined receive channels may form an input signal to the CW beamformer 110 that has a larger dynamic range compared to a configuration wherein only a single receive channel is used. The dynamic range of the input signal (to the CW beamformer 110) may be defined as the ratio between the maximum input level and the intrinsic voltage noise level of the received signal over the processing bandwidth.

Impedance matching circuitry, such as a transformer 278, is placed in the receive path (interconnected with the receive line 276) to transform the electrical impedance of the receive element 105 to maximize the dynamic range of the receivers 108 (i.e. the connected load) while achieving a desired or predetermined voltage noise level. Other component(s) may be used to form the impedance matching circuitry. In one embodiment, the transformation ratio, indicated on FIG. 3 as n:1, may be selected so that the transformed thermal noise from the electrical impedance of the receive element 105 is approximately 2 times the equivalent input noise of the receivers 108.

An inductor 280 may also be used in the receive path to tune out the capacitance of the parallel system channels 272-274, such as at a CW Doppler frequency f0 (i.e. the center operating frequency of the CW probe 106). Assuming that a per-channel input capacitance is Cin and that N channels are connected in parallel, the overall input capacitance is Npar*Cin. Equation 1 may be used to calculate the value of the inductor 280:

Ltune=1/(Npar*Cin*(2*pi*f0)̂2) Equation 1

Optionally, a capacitor may be placed in-line with each receive system channel 272-274, for example, capacitors 282 and 284 may be placed in-line with the forty-ninth and sixty-fourth system channels 272 and 274, respectively. The capacitors 282 and 284 are positioned downstream with respect to the receive line 276 and the receive element 105. The capacitors 282 and 284 may be used to block DC components if necessary, depending upon the design of the receivers 108. In some embodiments, series capacitance may already be included as a part of the input of the receivers 108.

FIG. 4 illustrates an exemplary miniaturized ultrasound system 132 that may be connected to the CW probe 106 through a port 136 that also accepts phased array probes. The transmit/receive circuitry 107 is provided within the CW probe 106 or optionally, may be provided within the system 132 as transmit/receive circuitry 109 (as shown in FIG. 1). A user interface 140 (that may also include an integrated display 142) is provided to receive commands from an operator. As used herein, “miniaturized” means that the ultrasound system 132 is a handheld or hand-carried device or is configured to be carried in a person's hand, pocket, briefcase-sized case, or backpack. For example, the ultrasound system 132 may be a hand-carried device having a size of a typical laptop computer, for instance, having dimensions of approximately 2.5 inches in depth, approximately 14 inches in width, and approximately 12 inches in height. The ultrasound system 132 may weigh about ten pounds, and thus is easily portable by the operator. The integrated display 142 (e.g., an internal display) is also provided and is configured to display a medical image.

The ultrasonic data may be sent to an external device 138 via a wired or wireless network 134 (or direct connection, for example, via a serial or parallel cable or USB port). In some embodiments, external device 138 may be a computer or a workstation having a display. Alternatively, external device 138 may be a separate external display or a printer capable of receiving image data from the hand carried ultrasound system 132 and of displaying or printing images that may have greater resolution than the integrated display 142.

FIG. 5 illustrates a hand carried or pocket-sized ultrasound imaging system 176 wherein the display 142 and user interface 140 form a single unit. By way of example, the pocket-sized ultrasound imaging system 176 may be a pocket-sized or hand-sized ultrasound system approximately 2 inches wide, approximately 4 inches in length, and approximately 0.5 inches in depth and weighs less than 3 ounces. A probe port 178 provides connection to scanning devices such as the CW probe 106 as well as phased array or other probes. The display 142 may be, for example, a 320×320 pixel color LCD display (on which a medical image 190 may be displayed). A typewriter-like keyboard 180 of buttons 182 may optionally be included in the user interface 140.

Multi-function controls 184 may each be assigned functions in accordance with the mode of system operation. Therefore, each of the multi-function controls 184 may be configured to provide a plurality of different actions. Label display areas 186 associated with the multi-function controls 184 may be included as necessary on the display 142. The system 176 may also have additional keys and/or controls 188 for special purpose functions, which may include, but are not limited to “freeze,” “depth control,” “gain control,” “color-mode,” “print,” and “store.”

The smaller sizes of the systems 132 and 176 limits the hardware that may be provided therein, as well as the number of probe ports. Having the transmit/receive circuitry 107 provided within the CW probe 106 allows an operator to connect the CW probe 106 to the phased array probe port. Alternatively, the transmit/receive circuitry 109 may be provided within the system 176.

FIG. 6 illustrates a console ultrasound imaging system 145 provided on a movable base 147. The console ultrasound imaging system 145 may also be referred to as a cart-based system. A display 142 and user interface 140 are provided and it should be understood that the display 142 may be separate or separable from the user interface 140. The user interface 140 may optionally be a touchscreen, allowing the operator to select options by touching displayed graphics, icons, and the like.

The user interface 140 also includes control buttons 152 that may be used to control the console ultrasound imaging system 145 as desired or needed, and/or as typically provided. The user interface 140 provides multiple interface options that the user may physically manipulate to interact with ultrasound data and other data that may be displayed, as well as to input information and set and change scanning parameters. The interface options may be used for specific inputs, programmable inputs, contextual inputs, and the like. For example, a keyboard 154 and track ball 156 may be provided.

The system 145 has at least one probe port 160 for accepting probes. A single port 160 may accept the CW probe 106 as well as phased array probes. The transmit/receive circuitry 107 needed to drive the CW probe 106 may be provided within the CW probe 106. This provides both a space and cost savings as the CW probe 106 may be used with a plurality of systems rather than being limited to the specific system having the dedicated CW probe port and transmit/receive circuitry installed therein. In another embodiment, the transmit/receive circuitry 109 may be provided within the system 145, supporting the connection of the CW probe 106 through the phased array probe port 160.

A technical effect of at least one embodiment is the ability to interconnect the CW Doppler probe with a phased array probe port on an ultrasound system. This allows the CW probe to be used with systems that do not have a dedicated CW Doppler probe port, as was previously required. The transmit/receive circuitry accomplishes the increased current and improved noise parameters, among other things, needed to operate the CW Doppler probe, and may be included within the CW Doppler probe. This configuration achieves a space and cost savings relative to the ultrasound system and facilitates the use of the CW Doppler probe with smaller-sized systems that may not have room for more than one probe port. Also, no additional transmit/receive circuitry needs to be installed within the ultrasound system. Alternatively, the transmit/receive circuitry may be included within the system, supporting the connection of the CW Doppler probe to the system through the same port that accepts phased array probes.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A stand-alone continuous wave (CW) Doppler probe (CW probe), comprising:
a scanning end comprising a single transmit element and a single receive element;
a connector having probe connecting elements configured to interface with port connecting elements of a probe port provided on an ultrasound system, the port connecting elements being configured to connect with system channels of the ultrasound system;
transmit circuitry for connecting a first set of the probe connecting elements in parallel, the transmit circuitry being interconnected with the transmit element; and
receive circuitry for connecting a second set of the probe connecting elements in parallel, the receive circuitry being interconnected with the receive element.

2. The CW probe of claim 1, wherein a number of the probe connecting elements within the first set is based on at least one of a level of current to drive the transmit element and a predetermined voltage noise level associated with one of the system channels.

3. The CW probe of claim 1, wherein a number of the probe connecting elements within the second set is based on a dynamic range associated with processing a receive signal detected by the receive element.

4. The CW probe of claim 1, wherein the probe port is configured to accept a connector of a phased array probe.

5. The CW probe of claim 1, the transmit circuitry further comprising:
a single transmit line connecting the first set of probe connecting elements and the transmit element; and
at least one circuit element configured to be in-line with at least one of the probe connecting elements within the first set of probe connecting elements, the first set of probe connecting elements being connected in parallel downstream of the at least one circuit element.

6. The CW probe of claim 1, the receive circuitry further comprising:
a single receive line connecting the receive element to the second set of probe connecting elements; and
impedance matching circuitry interconnected with the receive line for changing an impedance of the receive element with respect to a load interconnected with the second set of probe connecting elements through the probe port.

7. The CW probe of claim 1, the receive circuitry further comprising:
a single receive line connecting the receive element to the second set of probe connecting elements; and
a transformer interconnected with the receive line for changing an impedance of the receive element with respect to a load interconnected with the second set of probe connecting elements through the probe port.

8. The CW probe of claim 1, the receive circuitry further comprising:
a single receive line connecting the receive element to the second set of probe connecting elements; and
an inductor interconnected with the receive line for tuning out a capacitance associated with the system channels connected to the second set of probe connecting elements.

9. A stand-alone continuous wave (CW) Doppler probe (CW probe), comprising:
a scanning end comprising a single transmit element and a single receive element; and
a connector having a plurality of probe connecting elements configured to interface with a plurality of port connecting elements of a probe port provided on an ultrasound system, the probe port being configured to accept a connector of a phased array probe, the plurality of port connecting elements being configured to connect with system channels of the ultrasound system.

10. The CW probe of claim 9, further comprising:
a connector shell configured to enclose the connector; and
transmit/receive circuitry being formed within the connector shell of the CW probe, the transmit/receive circuitry connecting first and second sets of the probe connecting elements in parallel, wherein the first set of probe connecting elements is interconnected with the transmit element and the second set of probe connecting elements is interconnected with the receive element.

11. The CW probe of claim 9, further comprising transmit/receive circuitry for connecting first and second sets of the probe connecting elements in parallel, the first set of probe connecting elements being interconnected with the transmit element and the second set of probe connecting elements being interconnected with the receive element, wherein the transmit/receive circuitry is formed within the connector of the CW probe.

12. The CW probe of claim 9, further comprising transmit/receive circuitry for connecting first and second sets of the probe connecting elements in parallel, wherein the first set of probe connecting elements is interconnected with the transmit element and the second set of probe connecting elements is interconnected with the receive element, wherein each of the probe connecting elements is interconnected with a separate one of the system channels.

13. The CW probe of claim 9, further comprising transmit circuitry for connecting a first set of the probe connecting elements in parallel, wherein the first set of probe connecting elements is interconnected with the transmit element, wherein a number of the probe connecting elements within the first set is based on at least one of a level of current associated with the transmit element and a predetermined voltage noise level associated with a transmit signal on one of the system channels.

14. The CW probe of claim 9, further comprising receive circuitry for connecting a second set of the probe connecting elements in parallel, wherein the second set of probe connecting elements is interconnected with the receive element, wherein a number of the probe connecting elements within the second set is based on a dynamic range associated with processing receive signals detected by the receive element.

15. The CW probe of claim 9, further comprising transmit/receive circuitry for connecting first and second sets of the probe connecting elements in parallel, wherein the first set of probe connecting elements is interconnected with the transmit element and the second set of probe connecting elements is interconnected with the receive element, wherein the transmit/receive circuitry is formed within the ultrasound system.

16. A method for powering a stand-alone CW Doppler probe (CW probe) using a phased array connector, comprising:
determining respective areas of a single transmit element and a single receive element within a CW probe;
connecting N transmit channels in parallel within the CW probe to form a transmit line that is interconnected with the transmit element, N being based at least on the area of the transmit element; and
connecting M receive channels in parallel within the CW probe to form a receive line that is interconnected with the receive element, M being based at least on a dynamic range associated with processing receive signals from the receive element, the transmit and receive channels being connected through a probe port on an ultrasound system that is configured to interface with phased array probes.

17. The method of claim 16, wherein N is further based on a current level provided by a single transmit channel.

18. The method of claim 16, wherein M is further based on an available dynamic range of a single receive channel.

19. The method of claim 16, wherein the M receive channels are interconnected with a receiver within the ultrasound system through the probe port, the method further comprising transforming an electrical impedance of the receive element based on an input noise level of the receiver.

20. The method of claim 16, further comprising tuning out a capacitance of the M receive channels with an inductor having a value based on at least an input capacitance of a single receive channel and a center operating frequency of the CW probe.
